(19) United States
(12) Patent Application Publication
(10) Pub. No.: US 2025/0179417 A1
(43) Pub. Date: Jun. 5, 2025

(54) Continuous Manufacturing of a Biological Substance with Process Traceability

(71) Applicant: BiosanaPharma B.V., Leiden (NL)

(72) Inventors: Jacob Arthur Tijsterman, VIieland (NL); Jozef Anton Mari Pennings, Den-Haag (NL); Jeroen Frank Van De Laar, Leiden (NL)

(21) Appl. No.: 18/843,409

(22) PCT Filed: Mar. 1, 2023
(86) PCT No.: PCT/NL2023/050097

§ 371 Date: Sep. 3, 2024

(30) Foreign Application Priority Data
Mar. 1, 2022 (EP) 22159438.5

(51) Int. Cl. C12M 1/00; C12M 1/26

(52) U.S. Cl. CPC C12M 47/10; C12M 23/58; C12M 33/14; C12M 47/12

(57) ABSTRACT

The invention concerns a method and system for the continuous manufacturing of a biological substance with process traceability. The method and system involves performing a number of cell culture and harvest cycles using a bioreactor for producing a batch of the biological substance, with each cycle producing a sub-batch of the biological substance. For process traceability in consecutive cell culture and harvest cycles the produced sub-batches of biological substance are first collected in separate collectors before being continuously directed from each collector through downstream processing means for downstream processing of the biological substance to at least purify the biological substance into at least one lot containing purified biological substance.

The invention relates to bioreactors and cell culture contents and the use thereof in large scale manufacturing or production of biological substances, such as antibodies, from cell cultures cultured and/or maintained in a bioreactor. In the field of biological substance manufacturing a distinction can be made between batch manufacturing and continuous manufacturing. Generally, batch manufacturing involves production of the biological substance with a plurality of discrete process steps in a predefined follow order and usually involves some interruption between successive steps for quality testing of the intermediate products. As a result the complete production process can have lengthy residence times in the individual steps, especially the bioreactor, with relatively low production efficiency and/or product yields due to product degradation in time and process step inefficiency. A typical batch process has a duration of 3 weeks.

Continuous manufacturing instead relies on production of the biological substance with a more automated system that enables a mainly constant production and processing of the biological substance with only minimal or no interruption between the process steps and as a result is generally a more efficient production method.

With respect to quality control batch manufacturing and continuous manufacturing generally must meet the same or similar standards. Because continuous manufacturing is a more automated process it is better suited for early detection of irregularities or issues and may prevent or minimize failures from happening. However, presently a drawback of the production of biological substances using a continuous manufacturing process as opposed to the more conventional batch manufacturing lies in traceability, i.e. due to the continuous nature the intermediate and final products of a continuous process cannot easily and/or not adequately be separated and linked to allocated cell culture batches of the upstream process, including its raw materials, in process controls and related deviations, particularly as tracing methodologies for biological substances may not meet or be in accordance with current regulations. It may therefore be necessary when something in the manufacturing line is amiss resulting in a process failure to completely shut down the manufacturing process leading to product rejection, product shortages and increased costs.

It is therefore an aim to provide a method for the continuous manufacturing of a biological substance that overcomes this drawback. It is further an aim to provide a biological drug substance manufacturing system for the continuous manufacturing of a biological substance to this end. A particular aim is to provide such method and system that enables an efficient and cost-effective production of a biological substance, preferably by using cell cultures with high cell density.

In accordance in a first aspect a method is provided for the continuous manufacturing of a biological substance using a bioreactor or set of multiple bioreactors holding a culture of mammalian cells in culture medium for producing a batch of the biological substance, the method comprising performing a number of cell culture and harvest cycles, each cycle producing a sub-batch of the biological substance and comprising the steps of culturing cells, harvesting culture medium containing the biological substance while retaining cells in the bioreactor, filtering the culture medium containing the biological substance, and collecting in a collector, such as a harvest container, the sub-batch of biological substance, wherein in consecutive cell culture and harvest cycles the produced sub-batches of biological substance are collected in separate collectors, and wherein each sub-batch of biological substance, with its related raw materials, in process controls and deviations, is continuously processed from each collector through downstream processing means for downstream processing of the biological substance to at least purify the biological substance into at least one lot of biological substance containing purified biological substance.

Although in the field definitions for batch, sub-batch and lot may slightly differ, depending for instance on variations between different regulatory authorities in different jurisdictions, the following definitions are intended herein. ‘Batch’ means a specific quantity of a biological substance such as a drug or other material that is intended to have uniform character and quality, within specified limits, and is produced according to a single manufacturing order during the same cycle of manufacture. ‘Sub-batch’ means a part of the specific quantity of biological substance of the corresponding batch as produced during one culture and harvest cycle in one bioreactor. ‘Lot’ means a batch, or a specifically identified portion of a batch, having uniform character and quality within specified limits, or, in the case of a drug substance produced by continuous process, it is a specifically identified amount produced in a unit of time or quantity in a manner that assures it having uniform character and quality within specified limits. The problem of lack of traceability for continuous processes is that in view of residence time distributions for consecutive process steps it is not possible to provide a necessary separation in lots downstream of the process step which can be allocated to batches or sub-batches upstream of the process step. However, due to the collecting of consecutive sub-batches of the biological substance in at least two separate collectors in accordance with the first aspect herein the residence time distribution between such consecutive sub-batches in the downstream processing may be greatly reduced to no overlap at all or at most some minimal overlap limited to directly preceding and succeeding sub-batches. A minimal overlap between consecutive sub-batches according to the first aspect herein is at most 5% by volume of the total volume of the sub-batch with smallest total volume, but preferably the overlap, if any, is not more than 3% by volume of the total volume of the sub-batch with smallest total volume, more preferably not more than 1% by volume of the total volume of the sub-batch with smallest total volume.

This allows a separation of the sub-batches from each collector in confined and traceable downstream lots of biological substance. In case of a process failure a relatively small amount of defective material may be isolated, for example one or few lots or one or few sub-batches, resulting generally in less rejected product and a lower cost of production of the biological substance.

Preferably, the collecting of sub-batches of biological substance is performed alternatingly with two collectors. Alternating between two collectors provides the desired traceability with minimal equipment requirements for this purpose. More than two collectors may be employed as desired for further improvements on traceability where needed at the cost of requiring more equipment. Optionally, each sub-batch of biological substance is collected in the respective collector in a clarified form.

In a particular aspect of the method the steps of culturing cells, harvesting culture medium containing the biological substance while retaining cells in the bioreactor, and filtering the culture medium containing the biological substance is performed in consecutive cell culture and harvest cycles with different or separate bioreactors, each bioreactor arranged to hold a culture of cells in culture medium for producing a respective batch of the biological substance. Preferably, the consecutive cell culture and harvest cycles are performed alternatingly with two bioreactors. The coupling of multiple bioreactors to the multiple collectors provides for a continuous manufacturing and supply of biological substance sub-batches with a further improvement of production efficiency. For example, at least one of the bioreactors may remain in operation for performing a number of cell culture and harvest cycles and provide sub-batches of biological substance to at least one of the collectors for continuous downstream processing. The one or more other bioreactors may be used as desired, for example may be prepared for performing a number of cell culture and harvest cycles with a new or fresh cell culture. Preferably the bioreactors are controlled to provide in desired time intervals the sub-batches in any of the collectors, for example a first of the bioreactors may supply a sub-batch to a first of the collectors over a first time interval and a second bioreactor may supply a sub-batch to a second of the collectors over a second time interval, with the first and second time interval being consecutive, partly overlapping in time, or completely overlapping in time. It is preferred that for each bioreactor there is a corresponding collector configured for receiving sub-batches of the biological substance from its corresponding bioreactor only. However it is also possible to couple two or more bioreactors to one corresponding collector. For example instead of one relatively large bioreactor two or more relatively smaller bioreactors may be used. The two or more bioreactors may for instance each supply a quantity of biological substance or a sub-batch of biological substance to the corresponding collector, with each quantity or sub-batch being received in the collector consecutively, partly overlapping, or simultaneously.

Optionally in the method the downstream processing of each sub-batch of biological substance comprises a virus inactivation step and collecting each sub-batch of biological substance in a post-virus inactivation container. The collecting of consecutive sub-batches of biological substance in a post-virus inactivation container is preferably alternatingly performed with two separate post-virus inactivation containers. Optionally, the downstream processing of each sub-batch of biological substance comprises at least one of a multicolumn chromatography, cation exchange, anion exchange, nanofiltration, concentration and diafiltration step.

In a particular aspect the method involves for consecutive sub-batches performing any one of the multicolumn chromatography, cation exchange, anion exchange, nanofiltration, concentration and diafiltration processing steps alternatingly in dual set-up means configured for performing the respective processing step or steps.

For a completely continuous manufacturing process the method in a desired aspect involves supplying consecutive sub-batches of biological substance with two alternatingly controlled bioreactors to two connected collectors from which the respective sub-batches are continuously processed separately through the downstream process in dual set-up wherein the downstream processing means is selected from a virus inactivation means with post-virus inactivation container, multicolumn chromatography means, cation exchange means, anion exchange means, nanofiltration means, concentration means and diafiltration means. The dual set-up of downstream processing means may be provided in series or in parallel and are preferably controllable for alternatingly processing of consecutive sub-batches of the biological material.

Preferably, in the method, the culture and harvest cycle comprises incubating the cell culture in a bioreactor until the cell culture has reached the early stationary phase or stationary phase, and harvesting the culture medium with the biological substance by separating the cells from the culture medium containing the biological substance while keeping essentially all cells of the cell culture in the bioreactor for continued culture together with fresh culture medium.

Optionally, the cells used in the method are suspension cells, preferably CHO cells.

In a particular aspect the biological substance manufactured with the method is an antibody or an antigen binding fragment thereof.

In accordance with the aims of the invention in another aspect there is provided a biological drug substance manufacturing system for the continuous manufacturing of a biological substance comprising a bioreactor arranged to hold a culture of cells in culture medium for producing a batch of the biological substance, wherein the bioreactor is coupled to a separator arranged to receive a continuous flow of culture medium comprising cells and produced biological substance from the bioreactor and configured to retain the cells in the bioreactor and to direct the produced biological substance to a collector, such as a harvest container, of the system which collector has an inlet connected to the separator for receiving sub-batches of the biological substance from the bioreactor and having an outlet connected to downstream processing means of the system for continuous downstream processing of a sub-batch into at least one lot of biological substance, wherein the system comprises at least one further collector and wherein the collector and at least one further collector are configured for alternatingly receiving sub-batches of the biological substance and for flowing the respective sub-batches through the downstream processing means.

The biological drug substance manufacturing system is as such particularly suited for performing a method in accordance with one or more of the aspects described herein. In particular, the provision of at least two collectors enables the system to collect consecutive sub-batches of the biological substance in the at least two separate collectors and continuous downstream processing with greatly reduced overlap in the residence time distribution between such consecutive sub-batches. Thus, the system enables separation of the sub-batches from each collector in confined and traceable downstream lots of biological substance.

Preferably the system comprises at least two bioreactors each bioreactor arranged to hold a culture of cells in culture medium for producing a respective batch of the biological substance. Preferably, the consecutive cell culture and harvest cycles are performed alternatingly with two bioreactors. The consecutive cell culture and harvest cycles may be simultaneously performed at least in part, but preferably with at least the step of collecting the sub-batch of biological substance in the collector of each cycle being separated in time. The coupling of multiple bioreactors to the multiple collectors provides for a continuous manufacturing of biological substance with a further improvement of production efficiency. For example, at least one of the bioreactors may remain in operation for performing a number of cell culture and harvest cycles and provide sub-batches of biological substance to at least one of the collectors for continuous downstream processing. The one or more other bioreactors may be used as desired, for example may be prepared for performing a number of cell culture and harvest cycles with a new or fresh cell culture. Preferably, the bioreactors are controlled to provide in desired time intervals the sub-batches in one of the collectors, for example a first of the bioreactors may supply a sub-batch to a first of the collectors over a first time interval and a second bioreactor may supply a sub-batch to a second of the collectors over as second time interval, with the first and second time interval being consecutive, partly overlapping in time, or completely overlapping in time.

Optionally, the at least two bioreactors are connected to the collector and at least one further collector for flowing respective sub-batches of the biological substance from each bioreactor to at least one of the collectors.

In a particular aspect the system comprises control means arranged for controlled flow of sub-batches of the biological substance from each bioreactor to any of the collectors.

It is preferable that the downstream processing means of the system comprise at least two post-virus inactivation containers arranged to alternatingly collect consecutive sub-batches of biological substance from at least one of the collectors.

Particularly, the downstream processing means in the system comprise at least one of means for multicolumn chromatography, means for cation exchange, means for anion exchange, means for nanofiltration, means for concentration and means for diafiltration, wherein the at least one means is provided in dual set-up configured for alternatingly performing the respective processing step or steps on consecutive sub-batches of biological substance received from any of the collectors.

For a completely continuous manufacturing process the system in a desired aspect involves two alternatingly controlled bioreactors for supplying consecutive sub-batches of biological substance which are connected to two separate collectors each of the collectors connected to a respective separate downstream processing system for flowing the respective sub-batches continuously through the respective downstream processing system, each downstream processing system comprising at least one downstream processing means in dual set-up wherein the downstream processing means is selected from a virus inactivation means with post-virus inactivation container, multicolumn chromatography means, cation exchange means, anion exchange means, nanofiltration means, concentration means and diafiltration means. The dual set-up of downstream processing means may be provided in series or in parallel and are preferably controllable for alternatingly processing of consecutive sub-batches of the biological material.

In an aspect the method and system described herein may involve large scale culture or fermentation of a cell culture, preferably with a high density of cells. An advantage of a method and system of the invention is the high yield of the biological substance. The yield of substance is further increased with each further incubation/culture and harvest cycle. Thus preferably a method of the invention comprises at least 2, more preferably at least 5, more preferably at least 10 and preferably at least 20 incubation and harvest cycles.

The cell culture is preferably harvested with a harvest system that maintains many of the conditions of the incubation step. The harvest system therefore preferably comprises means for maintaining the culture temperature. When the volume of the cell culture in the bioreactor exceeds the capacity of the harvest system the entire cell culture can still be harvested. Preferably this is done by sequentially feeding clarified aliquots of the cell culture to the harvest system. The harvest cycle is then completed when essentially the entire cell culture is processed. The cell culture that is waiting to be processed is not mixed with cells that have already been processed by the harvest system. This is achieved by feeding the processed cells into a different bioreactor. The processed cells are therefore provided with fresh medium and fed into the different bioreactor for the subsequent incubation and harvest cycle. The cell density can be adjusted at this stage, if so desired. The cells can be concentrated or diluted, depending on the wishes of the operator. It is an advantage of the present invention that it is possible to implement a harvest system that has a different capacity than the volume of the cell culture.

An increase in the cell density is desirably, particularly in the first cycles, to quickly obtain the target cell density that is desired for production of the biological substance. Dilution is typically desired in later cycles to accommodate the continued increase in the number of cells.

The biological substance is preferably a substance that is excreted from the cells. The biological substance is preferably a protein, preferably an excreted protein. The protein preferably comprises more than 50, and preferably more than 100 amino acids. In a particularly preferred embodiment, the biological substance is a growth factor, an antibody or a soluble form of a membrane bound ligand. The biological substance can also comprise an antigen binding fragment of an antibody. Non-limited examples of such fragments are single chain Fv fragments, FAB-fragments, monovalent antibodies and heavy chain only antibodies. In a preferred embodiment the antibody is a mammalian antibody. In a preferred embodiment the antibody is a monoclonal antibody. In a preferred embodiment the antibody is a mouse, human or mouse/human chimeric antibody. The chimeric antibody is preferably a humanized murine monoclonal antibody or human antibody. Various types of humanization exist. Typically, at least the constant parts of the antibody are human.

Modern humanized antibodies also contain human variable regions wherein CDR regions are replaced by the murine CDRs. Alternatively, the antibody contains mutations in the murine variable regions to remove predicted human B and/or T cells epitopes. Presently it is possible to generate so-called fully human monoclonal antibodies. These are generated completely from human sequences. Immune response to these fully human antibodies are nevertheless still possible depending on the way of administration and the nature of the CDRs.

Cells can be grown either in suspension or adherent cultures. Some cells naturally live in suspension, without being attached to a surface, such as cells that exist in the bloodstream. There are also cell lines that have been modified to be able to survive in suspension cultures so they can be grown to a higher density than adherent conditions would allow. Adherent cells require a surface, such as tissue culture plastic or microcarrier, which may be coated with extracellular matrix components to increase adhesion properties and provide other signals needed for growth and differentiation. Adherent cells can typically be adapted to suspension growth, alternatively the cells can, as mentioned above, be adhered to microcarriers that are subsequently suspended and cultured in the bioreactor. The cells in the present invention are preferably cells that grow in suspension. Mammalian cells are preferred. Preferred production cells are the Chinese hamster ovary or CHO cells and the NSO cell line. In a particularly preferred embodiment the cells are CHO cells.

Cell growth in a typical fed-batch culture of mammalian cells can be classified into 7 stages: (A)lag phase; (B)early log phase; (C)log/exponential phase; (D)Early Stationery phase; (E)stationary phase; (G) Early Death phase (F)Death phase.

A. During lag phase, the cells adapt themselves to growth conditions. The cell numbers in the population do not increase.

B. During early log phase more and more cells have adapted and are entering the exponential phase.

C. Exponential phase (sometimes called the log phase or the logarithmic phase) is a period characterized by cell doubling. The number of new cells appearing per unit time is proportional to the present population. If growth is not limited, doubling will continue at a constant rate so both the number of cells and the rate of population increase doubles with each consecutive time period. For this type of exponential growth, plotting the natural logarithm of cell number against time produces a straight line. The slope of this line is the specific growth rate of the cells. The actual rate of this growth (i.e. the slope of the line in the figure) depends upon the growth conditions and the type of cells.

D. Early stationary phase (or early plateau phase) is the phase between the log-phase and stationary phase and is characterized by a progressive decline in the rate of population increase. This phase is characterized in that the growth rate of the population is a third or less from the maximum growth rate in the exponential phase. The early stationary phase ends when growth of the cell population is zero or less.

E. The stationary phase or plateau phase is often due to a growth-limiting factor; this is mostly depletion of a nutrient, and/or the formation of inhibitory products. In the stationary phase the size of the cell population remains more or less constant. Biological substance production is typically highest in this phase.

F. In the death phase, the cells run out of nutrients, or toxic products have build up to such an extend that the cells die.

G. The early death phase defines the end of the stationary phase when more and more cells die.

Depending on how the cells have been treated prior to initiating the culture the lag phase can be present or absent. For instance, large scale cultures are typically initiated from seed cultures and pre-cultures. In such cases it is possible to have a very short or absent lag phase, as the cells can have already adapted to the culture conditions in the fed-batch culture in an appropriate seed and/or pre-culture. In cyclic culture systems, such as the present invention, the later cycles typically repeat the stages C and D. Optionally one or more of the cycles include in addition the steps E, F and G. The exponential phase in later cycles may or may not reach the same maximum growth rate as the first cycle. In each cycle the start of the early stationary phase is characterized in that the growth rate of the population declines to a third or less from the maximum growth rate in the previous exponential phase. The growth rate is typically calculated from the tangent line of the growth curve.

In a particularly preferred embodiment the culture for each cycle is harvested while the culture is in stage C, D or E. Preferably the culture for each cycle is harvested while the culture is in stage C or D, more preferably at stage D. In another embodiment the culture in cycles 2 and further is harvested after at least one day, preferably at least two days, more preferably at least three days of incubation, irrespective of the stage of the stage of growth the cells are in at the time of initiation of the harvest step.

In another embodiment the culture for each cycle is harvested when a predetermined cell density has been reached. For cultures after the first culture and harvest cycle, it is preferred that the culture is harvested when the cells in the culture have reached a cell density of at least 0.5×10e8, preferably at least 1×10e8 and more preferably at least 2×10e8 per ml.

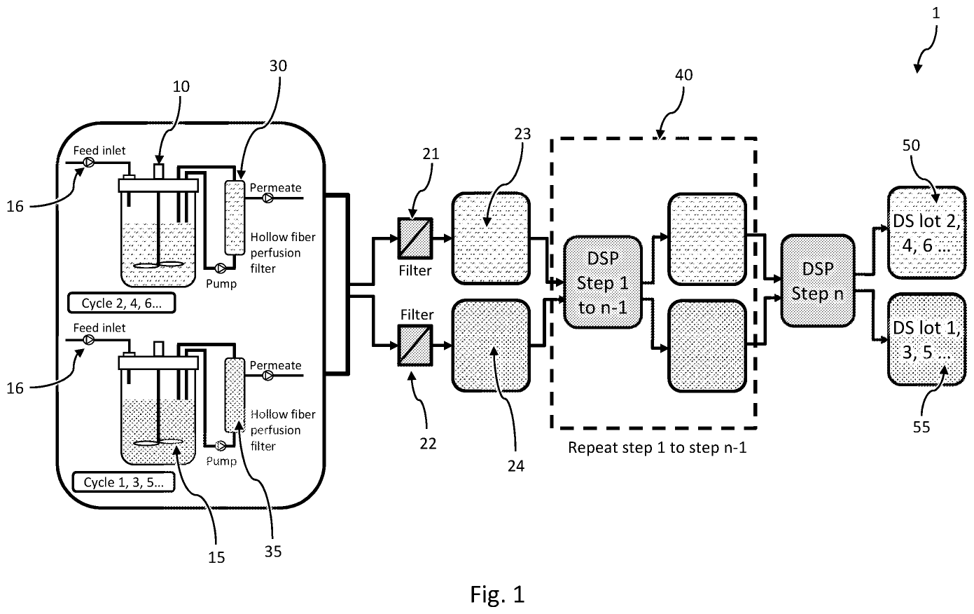

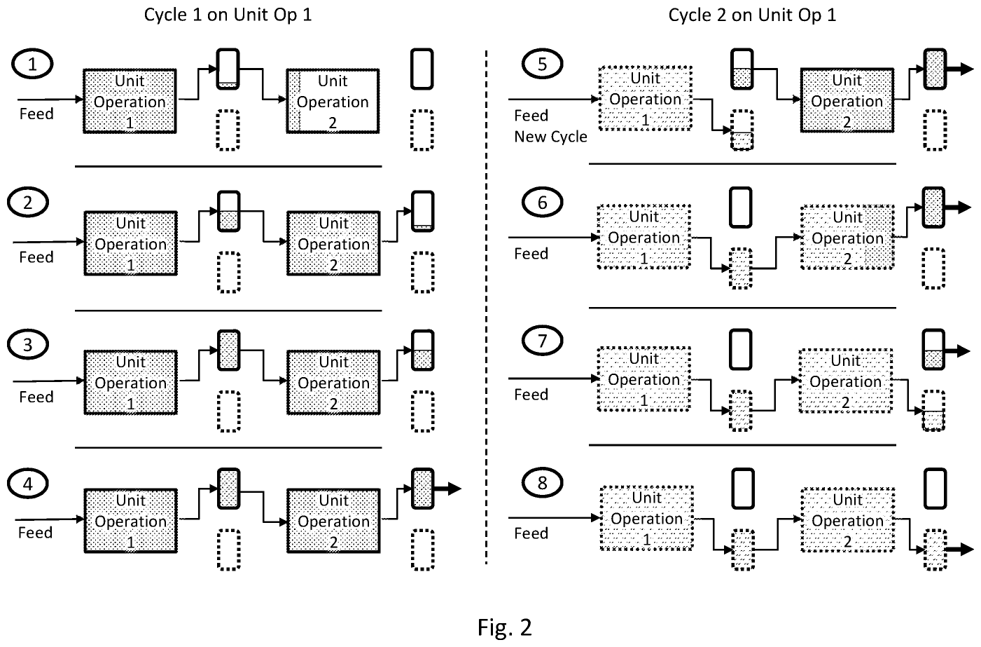

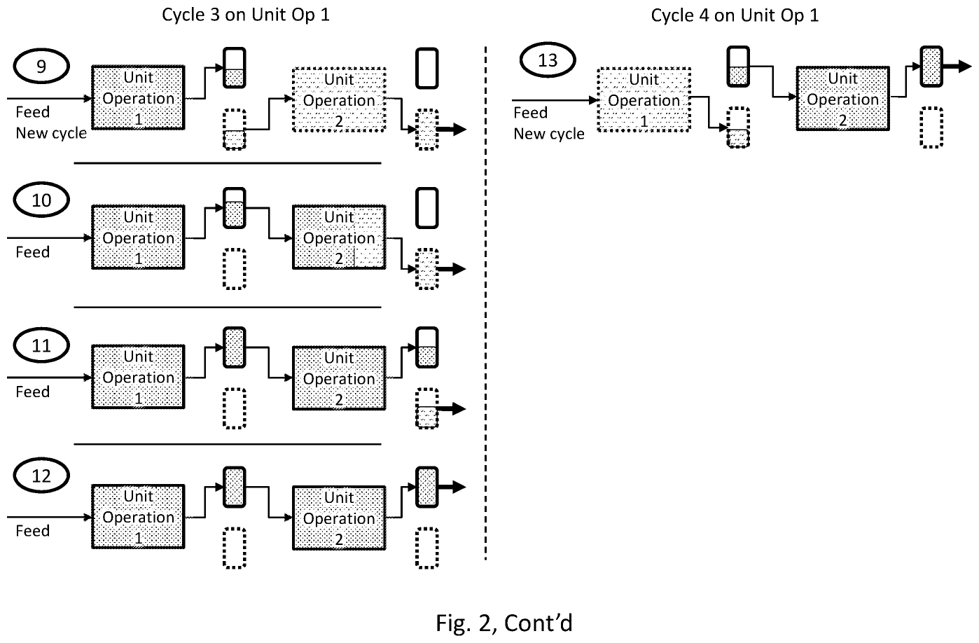

An example of a system for the production of a biological substance is given in FIG. 1 and FIG. 2. In the system, cells that produce said biological substance are cultured and are separated from a culture medium containing the biological substance. After separation, preferably essentially all cells are fed back into a bioreactor for continued culture together with fresh culture medium. In FIG. 1 a system 1 comprises a plurality of bioreactors 10, 15. The bioreactors are arranged to incubate a cell culture of cells suspended in a culture medium in said bioreactor. Each bioreactor 10,15 comprises a culture medium feed inlet 16 arranged to feed culture medium or a component thereof to the respective bioreactor to compensate for effluent loss. In particular, the feed inlet 16 may be directly coupled to the bioreactor, or may be coupled to a pump and switching unit, that connects to a separator 30 of the system. In addition, means may be provided for detecting a cell count or density in the bioreactors 10, 15. Typically, such means may be a cell count detector, density meter or flow cytometer, of a type known in the art. The detector can be arranged to detect a cell density. A predetermined threshold value can be determined heuristically or by means of a calculation for inputting to the detector, indicating that incubation of a cell culture in one of said bioreactors has reached the early stationary phase or stationary phase as hereabove clarified. A separator 30,35 is arranged communicatively between a bioreactor and a harvesting filter module 20. Thus each bioreactor 10,15 is connected to the harvesting filter module 20 via a respective separator 30,35. Each separator 30,35 comprises a hollow fiber perfusion filter which is supplied with cell culture that is pumped with a pump out of the respective bioreactor 10, 15 for separating product containing permeate from cells of the cell culture. The cells are maintained in the respective bioreactor while the product containing permeate is continuously filtered with membrane filter means 21,22 and collected in a respective collector 23,24 of the harvest filter module 20. Each culture and harvest cycle performed in one of the bioreactors 10,15 in this way provides a sub-batch of biological substance in a respective collector 23,24. The collectors preferably comprise or consist of a harvest bag 23,24. Each collector 23,24 is fluidically coupled to a downstream processing system 40 for continuous downstream processing of the sub-batches of biological substance into lots 50,55 of biological substance. The downstream processing system 40 comprises one or more processing means (not shown) selected from a virus inactivation means with post-virus inactivation container, multicolumn chromatography means, cation exchange means, anion exchange means, nanofiltration means, concentration means and diafiltration means. The one or more downstream processing means may be provided in dual set-up, for example in series or in parallel. As illustrated, the preferred set-up of the system involves a dual set-up of bioreactors (10,15) for providing sub-batches of biological substance, a dual set-up of collectors (23,24) for receiving sub-batches of biological substance from the respective bioreactor, a dual set-up of hold vessels (41, 42) for holding partially processed sub-batches of biological substance from the respective collectors, and a dual set-up of storage vessels (50,55) for storing lots of the biological substance. The system may comprise additional hold vessels in dual set-up for each or any of the processing steps.

In the preferred embodiment of the invention during incubation of the cell culture in the bioreactor at least part of the cell culture is continuously flown into the separation system that comprises a size selective membrane. However other separation means may alternatively or additionally be used. Cells are preferably maintained in the bioreactor after separation for a further culture and harvest cycle, but may optionally also be transferred into another bioreactor. With the latter setup it is possible to implement a harvest system that has a lower fluid capacity than the volume of the cell culture. In the further incubation it is preferred that at least part of the cell culture is continuously flowed into a separation system that comprises a size selective membrane. In the separation device consumed medium and substances (waste) with a lower molecular weight than the biological substance are removed from the culture. In the harvest system consumed medium, the biological substance and substances (wastes) having a higher molecular weight than the biological substance, such as cell debris, nucleic acids, and proteins are removed. This preferred embodiment is a particularly effective way in providing the conditions for multiple culture and harvest cycles at very high cell densities, while maintaining a relatively low cost system associated with the continuous culture in a bioreactor wherein at least part of the cell culture is continuously flowed into a separation system that comprises a size selective membrane.

It is preferred that culture conditions during culture are maintained in the separation system. The cultured cells experience culture conditions throughout the various culture and harvest cycles of the present invention. From the viewpoint of the cells, the culture is a continuous culture, whereas from a viewpoint of the process operator in charge of handling the waste disposal and harvesting of the biological substance, the culture is semi-continuous. From the viewpoint of the cells the system comprising the separation system, the centrifuge and the one or more bioreactors can be seen as one bioreactor for the culture of the cells. The cells experience culture conditions throughout the procedure. Not all culture conditions have to be maintained. A preferred culture condition to maintain is the temperature, another culture condition that is preferably maintained is oxygenation. Yet a further culture condition that is preferably maintained is the pH. Maintenance of a culture condition preferably means that the condition throughout the procedure does not deviate more than 20% from the value of the condition at the start of the procedure. Temperature is preferably maintained at 35.5-37.5° C., preferably at 37 C. The temperature may be lowered from a first temperature during culturing of cells towards a target cell density to a second temperature for inducing a steady rate of biological substance production with continuous media perfusion feed. Oxygenation is preferably maintained at 40-60% expressed as dissolved oxygen concentration, preferably at 50% dissolved oxygen concentration, pH is preferably maintained at a value of pH 6.7-7.5, preferably at pH 7 or 7.1.

An advantage of the method and system in accordance with one or more of the aspects and embodiments described herein is that continuous downstream processing is possible while maintaining some batch-like traceability thanks to the enabled separation between consecutive harvestings in two or more separate collectors. Biological substance can be continuously tapped from a respective collector for feed to the further downstream processing system. By processing in this way high product yields can be realized in a low volume continuous operational mode.

1. A method for the continuous manufacturing of a biological substance using a bioreactor holding a culture of mammalian cells in culture medium for producing a batch of the biological substance, the method comprising
performing a number of cell culture and harvest cycles, each cycle producing a sub-batch of the biological substance and comprising the steps of culturing cells, harvesting culture medium containing the biological substance while retaining cells in the bioreactor, filtering the culture medium containing the biological substance, and collecting in a collector, the sub-batch of biological substance,
wherein in consecutive cell culture and harvest cycles the produced sub-batches of biological substance are collected in separate collectors, and
wherein each sub-batch of biological substance is continuously directed from each collector through downstream processing means for downstream processing of the biological substance to at least purify the biological substance into at least one lot of biological substance containing purified biological substance.

2. The method according to claim 1, wherein the collecting of sub-batches of biological substance is performed alternatingly with two collectors.

3. The method according to claim 1, wherein each sub-batch of biological substance is collected in the respective collector in a clarified form.

4. The method according to claim 1, wherein in consecutive cell culture and harvest cycles the steps of culturing cells, harvesting culture medium containing the biological substance while retaining cells in the bioreactor, and filtering the culture medium containing the biological substance is performed with separate bioreactors, each bioreactor arranged to hold a culture of cells in culture medium for producing a respective batch of the biological substance.

5. The method according to claim 4, wherein consecutive cell culture and harvest cycles are performed alternatingly with two bioreactors.

6. The method according to claim 1, wherein the downstream processing of each sub-batch of biological substance comprises a virus inactivation step and collecting each sub-batch of biological substance in a post-virus inactivation container.

7. The method according to claim 6, wherein the collecting of consecutive sub-batches of biological substance in a post-virus inactivation container is alternatingly performed with two separate post-virus inactivation containers.

8. The method according to claim 1, wherein the downstream processing of each sub-batch of biological substance comprises at least one of a multicolumn chromatography, cation exchange, anion exchange, nanofiltration, concentration and diafiltration.

9. The method according to claim 8, wherein any one of the multicolumn chromatography, cation exchange, anion exchange, nanofiltration, concentration and diafiltration processing steps for consecutive sub-batches is alternatingly performed in dual set-up means configured for performing the respective processing step or steps.

10. The method according to claim 1, wherein the culture and harvest cycle comprises incubating the cell culture in a bioreactor until the cell culture has reached the early stationary phase or stationary phase, and harvesting the culture medium with the biological substance by separating the cells from the culture medium containing the biological substance while keeping essentially all cells of the cell culture in the bioreactor for continued culture together with fresh culture medium.

11. The method according to claim 1, wherein the biological substance is an antibody or antigen binding fragment thereof.

12. A biological drug substance manufacturing system for the continuous manufacturing of a biological substance comprising
a bioreactor arranged to hold a culture of cells in culture medium for producing a batch of the biological substance,
wherein the bioreactor is coupled to a separator arranged to receive a continuous flow of culture medium comprising cells and produced biological substance from the bioreactor and configured to retain the cells in the bioreactor and flow the produced biological substance to a collector of the system which collector has an inlet connected to the separator for receiving sub-batches of the biological substance from the bioreactor and having an outlet connected to downstream processing means of the system for continuous downstream processing of a sub-batch into at least one lot of biological substance,
wherein the system comprises at least one further collector and wherein the collector and at least one further collector are configured for alternatingly receiving sub-batches of the biological substance and for processing the respective sub-batches through the downstream processing means.

13. The system according to claim 12, comprising at least two bioreactors,
wherein each bioreactor is arranged to hold a culture of cells in culture medium for producing a respective batch of the biological substance.

14. The system according to claim 13, wherein the at least two bioreactors are connected to the collector and at least one further collector for flowing respective sub-batches of the biological substance from each bioreactor to at least one of the collectors.

15. The system according to claim 14, further comprising control means arranged for controlled flow of sub-batches of the biological substance from each bioreactor to any of the collectors.

16. The system according to claim 1, wherein the downstream processing means comprise at least two post-virus inactivation containers arranged to alternatingly collect consecutive sub-batches of biological substance from at least one of the collectors.

17. The system according to claim 1, wherein the downstream processing means comprise at least one of means for multicolumn chromatography, means for cation exchange, means for anion exchange, means for nanofiltration, means for concentration and means for diafiltration,
wherein the at least one means is provided in dual set-up configured for alternatingly performing the respective processing step or steps on consecutive sub-batches of biological substance received from any of the collectors.
